(19) United States
(12) Patent Application Publication
(10) Pub. No.: US 2026/0309703 A1
(43) Pub. Date: Oct. 8, 2026

(54) 3D-PRINTED CRISPR DIAGNOSTIC DEVICES AND USES THEREOF

(71) Applicant: University of Connecticut, Farmington, CT (US)

(72) Inventors: James F. Rusling, Storrs, CT (US); Hiniduma Liyanawitharanage Sunam Kesha Hiniduma, Holland, MA (US); Peyahandi Ishara Thilini De Silva, Newington, CT (US); Islam M. Mosa, Tanta (EG); Sherif Mohamed Shawky Abdou, Giza (EG)

(73) Assignee: University of Connecticut, Farmington, CT (US)

(21) Appl. No.: 19/638,907

(22) Filed: Apr. 3, 2026

Related U.S. Application Data
(60) Provisional application No. 63/783,287, filed on Apr. 4, 2025.

(51) Int. Cl. G01N 33/535; G01N 33/533

(52) U.S. Cl. CPC G01N 33/535; G01N 33/533

(57) ABSTRACT

The present disclosure describes a diagnostic platform that combines CRISPR/Cas technology with electrochemiluminescence (ECL) or fluorescence detection for highly sensitive and specific identification of disease biomarkers, such as microRNAs and proteins, without the need for complex amplification processes.

### RELATED APPLICATIONS

The instant application claims priority to U.S. Provisional Application No. 63/783,287, filed on Apr. 4, 2025. The entire contents of the foregoing application are expressly incorporated by reference herein.

### GOVERNMENT SUPPORT

This invention was made with government support under 2231490 awarded by the National Science Foundation. The government has certain rights in the invention.

### FIELD OF THE DISCLOSURE

The present disclosure provides a cutting-edge diagnostic platform that combines CRISPR/Cas technology with electrochemiluminescence (ECL) detection for highly sensitive and specific identification of disease biomarkers, such as microRNAs and proteins, without the need for complex amplification processes. The platform/system/device utilizes 3D-printed devices and advanced materials to enable multiplexed detection of biomarkers associated with conditions like Alzheimer's, cancer, and cardiovascular diseases, offering rapid, cost-effective, and accurate results. By leveraging CRISPR's precision and ECL's sensitivity, this platform has the potential to revolutionize clinical diagnostics, making it accessible for point-of-care use in hospitals, clinics, and even wearable devices.

### BACKGROUND

Accurate, multiplexed detection of disease-associated molecules such as nucleic acids, proteins, and small metabolites poses a significant challenge due to the complexity of biological samples and the vast diversity of potential targets. Conventional assays, including polymerase chain reaction and enzyme-linked immunosorbent assays, often demand time-consuming protocols, costly reagents, and specialized instrumentation that may not be feasible in resource-limited settings. Furthermore, these methods can lack the necessary sensitivity or specificity for certain low-abundance biomarkers, which are critical for the early diagnosis of disorders like cancer, cardiovascular diseases, neurodegenerative conditions, and infectious diseases. Techniques that harness the precision of gene-editing enzymes have been explored to overcome these issues, but fully achieving rapid, high-throughput, and cost-effective detection with minimal sample preparation remains a formidable task in the pursuit of reliable, point-of-care diagnostics.

### SUMMARY

The following brief summary is not intended to include all features and aspects of the present disclosure, nor does it imply that any claim must include all features and aspects discussed in this summary.

Disclosed herein are platforms/systems/devices and methods that combine CRISPR/Cas technology with electrochemiluminescence (ECL) detection for highly sensitive and specific identification of disease biomarkers, such as microRNAs and proteins, without the need for complex amplification processes.

In an aspect, disclosed is a platform/system/device for detection for highly sensitive and specific identification of disease biomarkers (such as microRNAs and proteins), the platform/system including CRISPR/Cas technology and electrochemiluminescence (ECL), without the need for any amplification step, as shown and described herein.

In an aspect, disclosed is a method for detection for highly sensitive and specific identification of disease biomarkers (such as microRNAs and proteins), the method includes a platform/system/device including CRISPR/Cas technology and electrochemiluminescence (ECL), without the need for any amplification step, as shown and described herein.

In some aspects, provided herein is a system for detection of biomarkers of clinical disorders and infectious diseases, the system comprising a component configured to house multiple CRISPR proteins and at least one sample, and a plate reader configured to detect fluorescence or electrochemiluminescence (ECL) from a reaction between the proteins and the at least one sample, without the need for any amplification, wherein the fluorescence or ECL is an indication of a biomarker of a clinical disorder or an infectious disease.

In some embodiments, the biomarkers comprise at least one of: nucleic acids, proteins, antibodies, or small peptides. In some embodiments, the CRISPR proteins comprise at least one of Cas13a, Cas12a, or Cas14. In some embodiments, the component comprises pyrolytic graphite electrodes coated with polyion films containing a ruthenium complex.

In some embodiments, the component comprises a microtiter plate. In some embodiments, the microtiter plate comprises 96 wells. In some embodiments, the microtiter plate is a 3D microtiter plate and wherein the microtiter plate comprises the CRISPR proteins immobilized on the 3D printed microtiter plate. In some embodiments, the microtiter plate is configured to house a CRISPR RNA corresponding to the CRISPR proteins, a buffer, a RNase inhibitor, and a fluorescent reporter. In some embodiments, the plate reader is configured to detect fluorescence produced by the fluorescent reporter. In some embodiments, each row of the microtiter plate includes one sample, a concentration of the sample increasing across each row.

In some aspects, provided herein is a method for detection of biomarkers of clinical disorders and infectious diseases, the method comprising collecting a sample from a subject, housing the sample within a component configured to house multiple CRISPR proteins, performing RNA extraction to get extracted RNA from the sample, and analyzing the extracted RNA using a plate reader configured to detect fluorescence or electrochemiluminescence (ECL) from a reaction between the proteins and the sample, without the need for any amplification, wherein the fluorescence or ECL is an indication of a biomarker of a clinical disorder or an infectious disease.

In some embodiments, the sample comprises 0.1-10% diluted plasma or serum.

In some embodiments, the method further comprises capturing and separating a target protein with a magnetic bead or monoclonal antibody. In some embodiments, the method further comprises resuspending the target protein with a nanoparticle functionalized with a secondary antibody and a RNA strand.

In some embodiments, the biomarkers comprise at least one of: nucleic acids, proteins, antibodies, or small peptides. In some embodiments, the CRISPR proteins comprise at least one of Cas13a, Cas12a, or Cas14.

In some embodiments, the method further comprises producing the component with a 3D printer. In some embodiments, the component comprises a microtiter plate, the microtiter plate produced with the CRISPR proteins immobilized on the microtiter plate.

In some embodiments, the RNA extraction is performed with a CRISPR RNA corresponding to the CRISPR proteins, a buffer, a RNase inhibitor, and a fluorescent reporter. In some embodiments, the plate reader is configured to detect fluorescence produced by the fluorescent reporter.

These and other aspects of the present invention are described in more detail below.

### BRIEF DESCRIPTION OF THE DRAWINGS

The subject matter, which is regarded as the disclosure, is particularly pointed out and distinctly claimed in the claims at the conclusion of the specification. The foregoing and other features, and advantages of the disclosure are apparent from the following detailed description taken in conjunction with the accompanying drawings in which:

FIG. 1 is a schematic diagram illustrating the concept of using CRISPR-Cas13a for the detection of miRNA biomarkers. The identification of the target miRNA biomarker in serum by the CRISPR-Cas13a leads to the activation of the CRISPR non-specific RNase activity which leads to the cleavage of the reporter molecule causing fluorescence emission. The absence of the target miRNA, leaves the CRISPR RNase inactive and hence the reporter molecule will not be cleaved leading to no fluorescence emission.

FIG. 2 depicts a CRISPR based liquid bioassay for the identification and quantification of miRNA panel for the early detection of Alzheimer Disease.

FIG. 3 depicts a mix and read CRISPR plate assay. A 96 well plate is designed and printed with specific dimensions. The cas13a protein is immobilized onto the plate and mixed with the corresponding crRNA (each target has its own pre-designed crRNA). Then, the reporter mixture (buffer, RNase inhibitor, the reporter) and the sample are added and mixed in each well and the generated fluorescent signal is read by the plate reader. Each row is designed to include one sample along with different concentration of the target to generate the standard curve for each sample.

FIGS. 4A-C are schematic diagrams illustrating the ECL concept of using CRISPR-Cas13a and poly-rG for the detection of miRNA biomarkers. After the target RNA hybridized with its corresponding crRNA, the cas13a cleave the target by the common cis-cleavage activity (FIG. 4A). Consequently, a conformational change in the cas13a occurred leading to activation of the non-specific collateral cleavage property, which in turn cleaves the poly-rG. The pyrolytic graphite working electrode fabrication by layer by layer assembly of PDDA/PSS/RuPVP, then, the working electrode added in the designed microfluidic chip with the Ag/AgCl and Pt as counter and reference electrodes respectively. Each spot on the working electrode array represents one sample and thus, multiplexing has been achieved (FIG. 4B). The assay procedures, the reaction buffer, RNase inhibitor, cas13a and crRNA is mixed and incubated at 37° C. for 12 minutes and then the target and the poly rG are added to the mixture ad loaded in the chip, ECL signal is generated, recorded & imaged by ChemiDoc CCD camera (FIG. 4C).

FIG. 5 is a representation of the different approaches followed by the invented platform in the protein detection by CRISPR/Cas technology. P-tau-181 protein is used here as a model. These approaches could be used for any other protein, antibodies, small molecules, and peptides.

FIG. 6 depicts designs of some microfluidic devices for portable and automated diagnostic sensors.

### DETAILED DESCRIPTION

Before the disclosed processes and materials are described, it is to be understood that the aspects described herein are not limited to specific embodiments, or examples, and as such can, of course, vary. It is also to be understood that the terminology used herein is for the purpose of describing particular aspects only and, unless specifically defined herein, is not intended to be limiting.

The present disclosure provides diagnostic platforms that combine CRISPR-based target recognition with fluorescence or electrochemiluminescence (ECL) signal generation for detection of biomarkers in biological samples. In various embodiments, the disclosed platforms are configured for direct detection of target analytes without nucleic-acid amplification.

Disclosed herein is a new multiplexed (i.e. multi-analyte) technology using CRISPR/Cas (CRISIPR is an abbreviation of Clustered Regularly Interspaced Short Palindromic Repeats which binds to a protein named Cas13a) technology as a disease detection tool with high sensitivity, specificity, and reproducibility. It can detect multiple types of disease biomarkers including nucleic acids, proteins, antibodies, small peptides, and other molecules. It can help to develop highly sensitive and selective biosensors for prognosis, early detection and follow up of treatment response for variety of clinical disorders such as cancer, cardiovascular diseases, neurodegenerative diseases, infectious diseases (viral, bacterial, fungal), liver and kidney function, and brain diseases. The disclosed technology that combines CRISPR/Cas systems (including Cas13a) with a 3D-printed platform and electrochemiluminescence (ECL) detection to identify multiple disease-related biomarkers in a single assay. In an aspect, the platform/system immobilizes Cas and guide RNAs in microtiter plates or on pyrolytic graphite electrodes coated with polyion films containing a ruthenium complex, generating a quantifiable ECL signal upon activation by target molecules such as microRNAs or proteins. The platform/system employs functionalized nanoparticles and magnetic beads for protein detection and can measure unamplified targets directly from clinical specimens, reducing complexity and cost. Its modular design supports integration with various devices—including microfluidics, possibly wearable sensors, and smartphone-based systems—and achieves femtogram/mL-level sensitivity for different biomarkers. In some embodiments, the platform/system is built on the SHERLOCK platform, it features multiplexing capabilities, efficient sample preparation, and straightforward validation protocols, enabling broad potential for clinical diagnostics, including early detection of neurodegenerative diseases and cancer.

Although CRISPR-based detection and 3D-printed microfluidic devices have been separately reported, the direct, unamplified detection of multiple biomarkers using Cas systems integrated into 3D-printed plates has not been achieved. Further, ECL assays based on ruthenium complexes are known, but coupling them directly with CRISPR enzymes (Cas13a/Cas12a) in a multiplexed platform for unamplified nucleic acid and protein detection has not been studied, for example, the existing CRISPR-based assays typically use fluorescent or lateral flow readouts rather than ECL. Prior CRISPR-based methods (such as SHERLOCK and DETECTR) primarily focus on nucleic acids. The disclosed platform/system and method use a novel integration of functionalized nanoparticles and magnetic beads to detect proteins within the same CRISPR/ECL workflow allowing simultaneous analysis of varied biomarkers. In some embodiments, the platform/system includes a combination of a 3-sensor electrode design with CRISPR-driven cleavage and imaging in the fg/mL range. While immunomagnetic separation of p-tau (Tau protein) is used in neurological research, the described approach converts protein binding events into a CRISPR-detectable RNA trigger (via a miRNA conjugate) thereby combining CRISPR collateral cleavage with antibody-functionalized nanoparticles specifically for protein targets.

In an embodiment, the platform/system and method disclosed herein enables comprehensive, multiplex detection of disease biomarkers, offering rapid and highly sensitive testing for conditions such as cancer and neurodegenerative diseases. In an embodiment, the platform/system integrates with portable or wearable devices, facilitating quick, on-site diagnostics for resource-limited settings (point of care testing). In an embodiment, the platform/system provides a highly sensitive platform for biomarker discovery, validation, and therapeutic monitoring. In an embodiment, the platform/system identifies and tracks predictive biomarkers to guide personalized treatment strategies and monitor patient response to therapies. In an embodiment, the platform/system and method offer the potential for user-friendly, cost-effective at-home testing kits (direct to consumer diagnostics), leveraging minimal sample preparation and multiplex capabilities.

The platforms/systems and methods of the present invention provide the following advantages:
1. High sensitivity compared to Real-Time PCR and Sherlock methods: This CRISPR/Cas-based ECL platform can directly detect unamplified DNA or RNA targets with femtogram/mL-level limits of detection, whereas Real-Time PCR and Sherlock typically requires amplification steps to reach the required sensitivity.
2. Cost-effectiveness relative to ELISA: By integrating Cas proteins onto 3D-printed devices, the invention reduces the need for costly enzymes, antibodies, and equipment required for ELISA assays.
3. Versatile detection across multiple biomolecules: Unlike standard CRISPR-based methods like SHERLOCK that focus predominantly on nucleic acids, this platform also detects proteins and antibodies on a single device.
4. Multiplexed analyses in a single run: In contrast to standard single-analyte SHERLOCK or PCR tests, the platform uses multiple embedded sensors, enabling simultaneous detection of multiple disease biomarkers in one assay.
5. Reduced turnaround time compared to typical laboratory methods: By eliminating lengthy amplification or incubation steps, results can be obtained more rapidly than with standard Real-Time PCR, SHERLOCK, or ELISA procedures.
6. Minimal specialized equipment needs: The use of portable 3D-printed microtiter plates and ECL systems makes the approach more accessible than high-end PCR machines or plate readers required for conventional tests.
7. Improved specificity through CRISPR collateral cleavage: The Cas13a-based platform/system leverages the collateral RNA cleavage mechanism, offering a highly specific signal that reduces false positives more effectively than many standard immunoassays.
8. High adaptability for point-of-care applications: Unlike many conventional diagnostic devices that remain confined to centralized labs, this invention's compact and modular design allows for point of care use, and integration with wearable sensors and smartphone-based detection.
9. Scalability for broad clinical use: Building on CRISPR and ECL techniques, the technology can be extended from small research labs to large diagnostic centers, surpassing the throughput limitations of methods like traditional ELISA or single-plex PCR assays.

### Platform/System/Device for Detecting Disease Biomarkers

In an aspect, disclosed is a platform/system or device for identification of disease biomarkers, the platform/system/device including CRISPR protein (for example, cas13a, Cas12a, Cas14, any other suitable CRISPR associated proteins, or any combinations thereof); and an electrochemiluminescence (ECL) detection system.

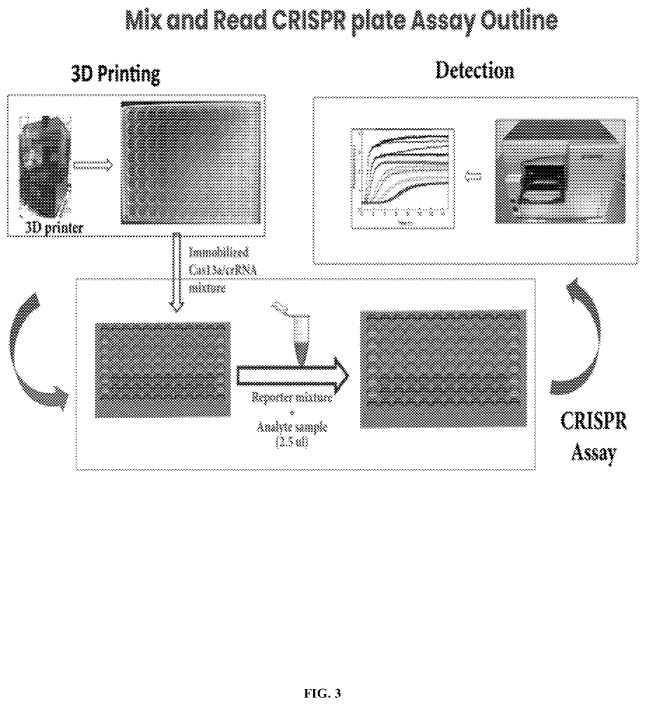

In an embodiment, the platform/system includes CRISPR cas13a, Cas12a, Cas14, any other suitable CRISPR associated proteins, or any combinations thereof to detect proteins, peptides, nucleic acids (coding and non-coding RNAs, single stranded and double stranded DNA), which are well-established biomarkers for any clinical disorders and infectious diseases, without pre-amplification step. In some embodiments, the platform/system can also detect other biomarkers to follow up response of treatment. In an embodiment, the platform/system includes Fluorescent based signal that is designed to mix the sample with a ready-made 3D printed 96 microtiter plate and read the signal. In an embodiment, the platform/system includes a cas protein that is immobilized onto the detection chamber along with its corresponding CRISPR RNA (with high stability and functionality) and a buffer, RNase inhibitor, and a fluorescent reporter; the sample is added to this platform/system, and the generated signal is recorded as explained in FIG. 3.

In an aspect, disclosed is a platform for identification of disease biomarkers, the platform/system/device including CRISPR protein (for example, cas13a, Cas12a, Cas14, any other suitable CRISPR associated proteins, or any combinations thereof); and fluorescence detection system. In an embodiment the platform further includes fluorescence plate reader or similar device capable of measuring fluorescent in multiple well of a multiwall plate.

The platform/system including Electrochemiluminescent (ECL) based signal-fluorescence with electrochemical source, doesn't need external light, this fluorescence without any light source is designed by utilizing for the first time the redox reaction between Ruthenium complexes such as bipyridyl poly vinyl pyridine [Ru(bpy)₂ (PVP)₁₀]⁺² and guanine nucleotides in generating ECL signal by CRISPR/cas technology. This is achieved by firstly construction a layer-by-layer assembly of poly[diallyl dimethyl ammonium chloride](PDDA), polystyrene sulfonate (PSS) and Ru PVP on pyrolytic graphite sheet as working electrode and then, the sample, buffer, RNase inhibitor, cas13a crRNA, poly r Guanine (having ribonucleic acid backbone, poly-rG), is added and the ECL signal imaged by CCD camera. The principle of the detection is that in the presence of the target, cas protein will be activated, which in turn will cleave the poly-rG generating single guanine nucleotides which will be oxidized by Ru⁺³ and electronically excited Ru⁺² is generated that emits light at about 610 nm upon electronic relaxation.

The platform/system is designed to detect proteins, peptides, and antibodies. For the protein detection, a nanoparticle (gold, silica, silver, copper, titanium nanoparticles or silica dopped ruthenium nanoparticle), is functionalized with both polyclonal antibody specific to the target protein and RNA strand (which acts as a target to a crRNA), and Magnetic bead functionalized with monoclonal antibody (capture antibody). First, the protein target is captured and separated by the magnetic bead/monoclonal Ab and then resuspended with the other nanoparticle functionalized with secondary Ab and RNA strand. The immune complex formed and hybridization of the RNA on the nanoparticles with its corresponding crRNA will lead to activation of the cas protein and a fluorescent or ECL signal will be generated according to the method applied.

This new platform/system allows the multiplexing and simultaneous detection of biomolecules (nucleic acids/proteins/peptides/antibodies, or any combinations thereof) using CRISPR/Cas for the first time which is a significant improvement in the health care diagnostics. The disclosed systems may be configured for multiplex analysis of biomarkers associated with neurodegenerative disease, cancer, cardiovascular disease, infectious disease, and other conditions. In an embodiment, the platform/system can be used for detection of panels of microRNAs and protein biomarkers for early diagnosis of neurodegenerative conditions such as Alzheimer's disease, Parkinson's disease, Amyotrophic Lateral Sclerosis (ALS), Huntington's disease, and/or various types of dementia. In an embodiment, the platform/system can be used for detection of panels of microRNAs and protein biomarkers for early diagnosis of Alzheimer's disease.

### Method for Detecting Disease Biomarkers

In an aspect, disclosed is a method for detection for highly sensitive and specific identification of disease biomarkers (such as microRNAs and proteins), the method includes a platform/system including CRISPR/Cas technology and electrochemiluminescence (ECL), without the need for any amplification step. In an embodiment, the method includes collecting a sample from a subject; performing RNA extraction to get extracted RNA; and analyzing the extracted RNA using the platform including CRISPR/Cas technology and electrochemiluminescence (ECL), without the need for any amplification step. In an embodiment, the method includes collecting a sample from a subject; and analyzing the sample (for example, plasma or serum) using the platform including CRISPR/Cas technology and electrochemiluminescence (ECL), to detect desired proteins in the sample. In an embodiment, for protein detection, the sample can be about 0.1-10% diluted plasma/serum and other body fluids. For example, for protein detection, the sample can be about 0.1, 1, 2, 3, 4, or 5% plasma/serum and other body fluids diluted in buffer. In an embodiment, the method includes non-coding RNAs, such as long-noncoding RNA, and exosomal RNAs (coding and non-coding). In some embodiments, the method includes a panel for each disease to be detected for non-coding and coding, and exosomal cargo (proteins and RNAs). In an embodiment, the subject is a mammal such as human, cat, dog, pig, or horse. In an embodiment, the subject is human.

In an aspect, disclosed is a method for detection for highly sensitive and specific identification of disease biomarkers (such as coding and non-coding RNA) and viral RNA and proteins), the method includes a platform/system including CRISPR/Cas technology and fluorescence detection, without the need for any amplification step. In an embodiment, the method includes collecting a sample from a subject; performing RNA extraction to get extracted RNA; and analyzing the extracted RNA using the platform including CRISPR/Cas technology and fluorescence detection, without the need for any amplification step. In an embodiment, the method includes collecting a sample from a subject; and analyzing the sample (for example, plasma, serum, urine, and other body fluids) using the platform including CRISPR/Cas technology and fluorescence, to detect desired proteins in the sample. In an embodiment, for protein detection, the sample can be about 0.1-10% diluted plasma/serum and other body fluids. For example, for protein detection the sample can be about 0.1, 1, 2, 3, 4, or 5% diluted plasma/serum, and other body fluids. In an embodiment, for protein detection, the analyte protein is labeled in some specific way with an RNA strand complementary to a crRNA in the CRISPR detection system, e.g., using a specific antibody and/or nanobody for the protein that is labeled with the RNA strand complementary to a crRNA in the CRISPR detection system. In an embodiment, the subject is a mammal such as human, cat, dog, pig, or horse. In an embodiment, the subject is human.

### Kit for Detecting Disease Biomarkers

In an aspect, disclosed is a kit for detection for highly sensitive and specific identification of disease biomarkers (such as microRNAs and proteins), the kit includes a platform/system including CRISPR/Cas technology and electrochemiluminescence (ECL), without the need for any amplification step, and instructions to use the kit. In an aspect, disclosed is a kit for detection for highly sensitive and specific identification of disease biomarkers (such as microRNAs and proteins), the kit includes a platform/system including CRISPR/Cas technology and fluorescence detection, without the need for any amplification step, and instructions to use the kit.

In a related aspect, the present disclosure provides a kit for implementing a method for detection for highly sensitive and specific identification of disease biomarkers (such as coding and non-coding RNAs and proteins) in a sample. The kit includes instructions for performing the method. For example, the kit includes instructions for (a) collecting a sample from a subject; and (b) processing the sample collected in step (a) using the platform/system described herein; and (c) detecting the target disease biomarkers in the sample.

In another aspect, the present disclosure provides a kit comprising a platform/system disclosed herein, the kit comprising: (a) a CRISPR/Cas technology; and (b) electrochemiluminescence (ECL). In another aspect, the present disclosure provides a kit comprising a platform/system disclosed herein, the kit comprising: (a) a CRISPR/Cas technology; and (b) fluorescence detection.

### CRISPR Based Detection of Protein Biomarkers (Fluorescent and ECL Based Signals):

Protein biomarkers are widely used as important diagnostic tool for most of human diseases. The CRISPR technology can detect proteins in blood, serum, plasma, saliva, cerebrospinal fluid, cells, or tissues.

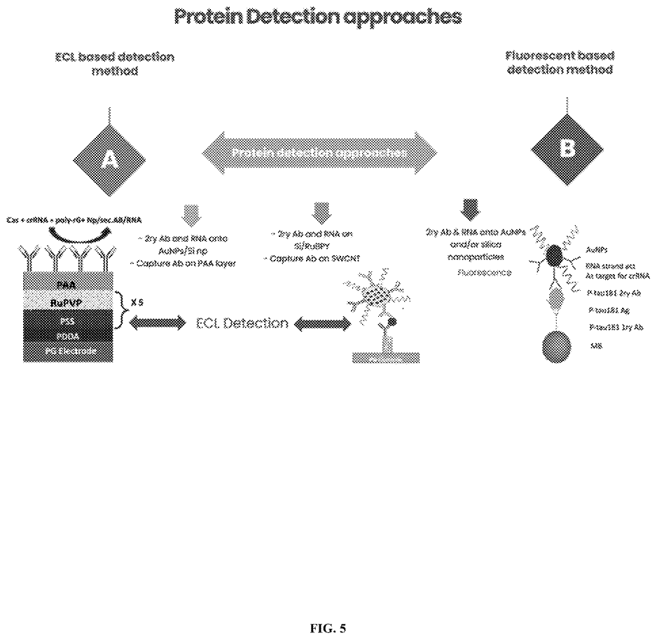

The different approaches developed for proteins biomarkers detection by CRISPR/Cas technology are summarized in FIG. 5.

A sample containing protein biomarker is first captured by Magnetic Bead (MB) functionalized by monoclonal antibody against the protein of interest (p-tau-181 as a model), and separated by a magnet, which is then resuspend in nanoparticle solution, which are functionalized with both secondary polyclonal antibody and RNA strand which acts as a target for the crRNA. The immune complex formed (MB/mAb/Ag/np/sec. Ab/RNA) is separated by a magnet and resuspended in the reaction buffer/reporter RNA molecule and added to the immobilized crRNA/Cas13a and the fluorescence reading is recorded. In the presence of the protein (antigen), the immune complex will be formed, and the RNA attached to the nanoparticles will hybridize to the crRNA activating the cas 13a protein which led to cleavage of the reporter RNA molecule generating fluorescence. On the other hand, in the absence of the protein (antigen), no immune complex will be formed and thus, no fluorescence signal detected. The nanoparticles that are functionalized with secondary polyclonal antibody and the RNA strand could be: gold, silver, silica, titanium.

The ECL based detection approach is achieved through firstly, functionalization of the fabricated working electrode by a capture antibody at the outer layer after layer by layer assembly of PDDA/PSS/Ru-PVP/PSS. The reaction is initiated by first incubating the sample contains the protein of interest on the working electrode, wash and then add the crRNA/Cas13a, reaction buffer, poly rG and the nanoparticle functionalized with secondary polyclonal Ab and the RNA strand. The ECL signal is generated if the RNA strand hybridizes with the crRNA, activating the cas13a which in turn will cleave the poly rG producing guanines nucleotides, which react with Ru-PVP according to the reaction pathway in equations 1-4. Secondly, homemade Single Walled Carbon Nanotube (SWCNT), were assembled on pyrolytic graphite working electrode and then, capture monoclonal antibody is conjugated with the carboxyl functionalized SWCNT via EDC/NHS chemistry. Then, the sample contains the protein target is incubated with working electrode (PG/SWCNT/mAb). The ECL bioconjugate (Ruthenium dopped silica nanoparticles functionalized with secondary polyclonal Ab and RNA strand, the latter acts as target for the crRNA) is then, added to the working electrode along with crRNA/Cas13a, reaction buffer, RNase inhibitor and poly rG. In the presence of the target, the immune complex will be formed and the ECL signal is produced as discussed above. The ECL light intensity is integrated by CCD camera.

The reaction includes but not limited to the following components: Primary antibody, nanoparticles (gold, silica, ruthenium doped silica, silver nanoparticles) linked to secondary antibody and target RNA or DNA sequence, poly rG, nuclease assay buffer (40 mM Tris-HCl, 60 mM NaCl, 6 mM MgCl2, pH 7.3), The assay contains 20-100 nM purified LwCas13a (or C2C2), 5-50 nM CRISPR-RNA, 10-200 nM quenched fluorescent RNA reporter molecule from Thermo Scientific known as RNAse Alert v2, and 2 μL murine RNase inhibitor (New England Biolabs). CRISPR associated protein (Cas proteins) including but not limited to either Cas13a (C2C2), Cas12a, Cas14, C2c1, C2C3, or any combinations thereof.

The primary antibodies may be immobilized on the sensor surface by adsorption, covalent bonding, oriented on nanoparticles, protein A, chitosan, or other polymers.

The analyte may be captured on the surface by a specialized receptor or using oligonucleotide or peptide molecule or nanobody replacing the use of primary antibodies.

The secondary antibodies may be replaced by a specialized receptor or using oligonucleotide or peptide molecule.

The effector protein may be Cas13a known as C2C2, or Cas12a, or C2c1 or C2c3, Cas14, or any suitable CRISPR associated protein that develops non-specific RNase OR DNase activity after binding to the target nucleic acid.

### Detection of Peptides, Polypeptides, and Small Molecules Using CRISPR System

The above CRISPR assay can be adopted to detect small molecules, drugs, and peptides in circulating blood, serum, plasma, saliva, urine, CSF to detect various diseases or screen drug toxicity. This can be achieved by binding a primary antibody or a receptor to the sensor surface specific to one or more of the above molecules followed by a similar protocol as described in the protein section of FIG. 5, but with changing the type of antibodies to the one specific for the target molecule being detected. Receptors or aptamers may also be used to replace the antibodies for capturing the target analyte.

### Detection of Antibodies Using CRISPR System

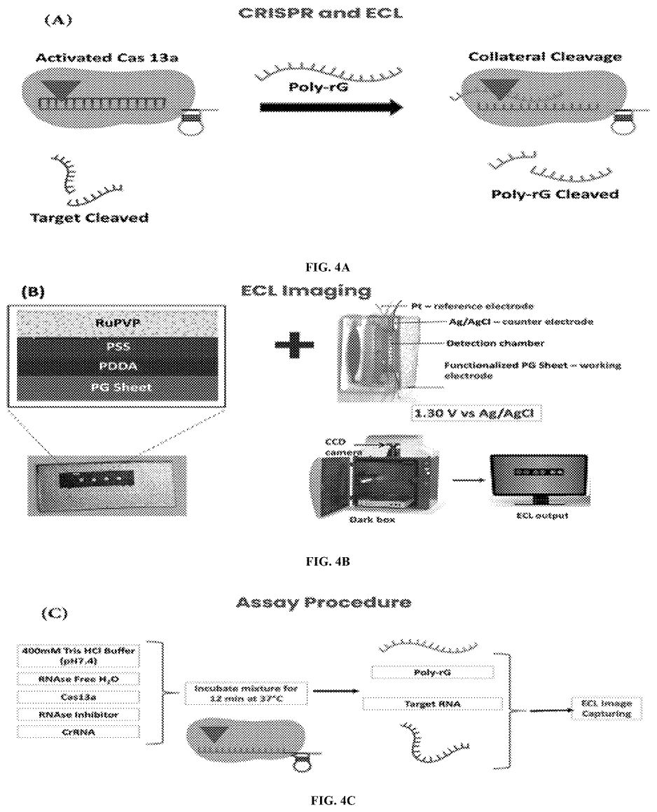

The detection of antibodies in body fluids such as blood is of clinical significance to detect the viral and bacterial infection. This can be achieved by capturing a viral antigen on the sensor surface, followed by incubating the patient's blood sample on the sensor surface to capture the antibody specific to the viral antigen of interest. A nanoparticle decorated with Anti-IgG and with the CRISPR target RNA or DNA can then bind to the antibody from the sample on the surface as seen in FIG. 4. By addition of the CRISPR Cas13a, Cas12a or Cas14 systems, buffer, reporter molecule, poly rG as described above will lead to the fluorescent or ECL detection signal.

### Device and Instrumentation for CRISPR Based Protein, Peptide, Polypeptide, Small Molecule, and Drug Detection

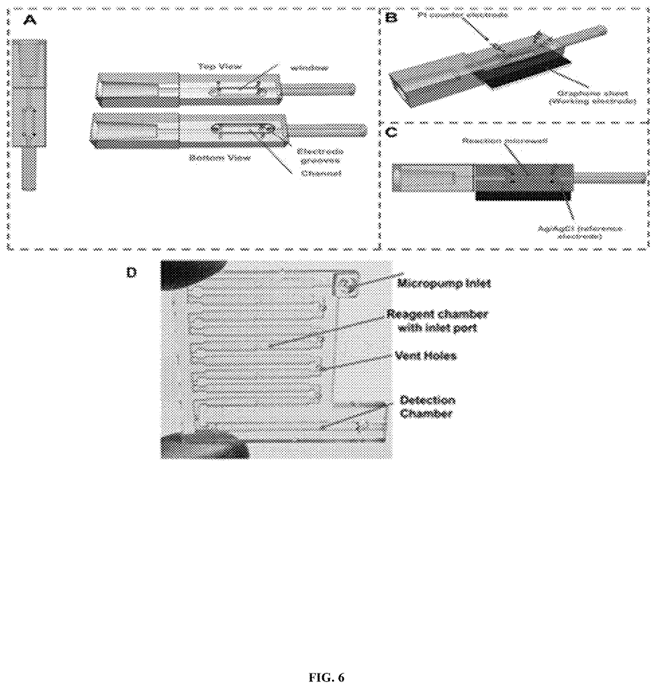

The devices used in this diagnostic platform could be 3D printed microfluidic devices such as those printed by formlab1, 2, and 3 printers. The microfluidic device can contain channels to load different reagents and be connected to automatic pump or include a vacuum champers to drive the reagents through the device. The device could be a paper microfluidic, chip microfluidic device, electrochemical sensor, electro chromic chip, 96 well plate, or ELISA plates. Two example designs are demonstrated in FIG. 6.

The systems described herein can be embodied on diagnostic devices. Several substrates and configurations of devices capable of defining multiple individual discrete volumes within the device may be used. Different suitable materials and device designs can be employed to create multiple separate featured/specific volumes within the device.

As used herein “individual discrete volume” refers to a discrete space, such as a container, receptacle, droplet, or other arbitrary defined volume or space that can be defined by properties that prevent and/or inhibit migration of target molecules, for example, a volume or space defined by physical properties such as walls, for example, the walls of a well, tube, or a surface of a droplet, which may be impermeable or semipermeable, or as defined by other means such as chemical, diffusion rate limited, electro-magnetic, or light illumination, or any combination thereof that can contain a target molecule and a indexable nucleic acid identifier (for example, nucleic acid barcode). By “diffusion rate limited” (for example, diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example, gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge, or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example, poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion. In some embodiments, any of the applications, methods, or platforms/systems described herein requiring exact or uniform volumes may employ the use of an acoustic liquid dispenser.

In certain embodiments, the platform/system/device comprises a flexible material substrate on which several detection spots may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g., multiple clinical samples from different individuals) for the presence of the same target, or a limited number of targets, or aliquots of a single sample (or multiple samples from the same source) for the presence of multiple different targets in the sample.

In some embodiments, a dosimeter or badge may be provided that serves as a sensor or indicator such that the wearer is notified of exposure to certain microbes or other agents. For example, the systems described herein may be used to detect a particular pathogen. Likewise, aptamer-based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous microbes as quickly as possible, for example, for biological or chemical warfare agent detection. In some embodiments, such a surveillance badge may be used for preventing exposure to dangerous microbes or pathogens in immunocompromised patients, burn patients, patients undergoing chemotherapy, children, or elderly individuals.

Samples sources that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an embodiment, the environmental sample is taken from a solid surface, such as a surface used in the preparation of food or other sensitive compositions and materials.

In some embodiments, the elements of the systems described herein may be placed on a single use substrate, such as swab or cloth that is used to swab a surface or sample fluid. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the CRISPR systems are designed to detect, for example identifying proteins that may be used to identify a suspect, or certain tissue or cell markers to determine the type of biological matter present in a sample. Likewise, the single use substrate could be used to collect a sample from a subject/patient—such as a saliva sample from the mouth—or a swab of the skin. In other embodiments, a sample or swab may be taken of a meat product in order to detect the presence of absence of contaminants on or within the meat product.

Near-real-time microbial diagnostics are needed for food, clinical, industrial, and other environmental settings. In certain embodiments, the present invention is used for rapid detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulnificus, Yersinia enterocolitica and Yersinia pseudotuberculosis, Brucella spp., Corynebacterium ulcerans, Coxiella burnetii, or Pie siomonas shigelloides).

In certain embodiments, the device is a microfluidic device that generates and/or merges different droplets (i.e. individual discrete volumes). For example, a first set of droplets may be formed containing samples to be screened and a second set of droplets formed containing the elements of the systems described herein. The first and second set of droplets are then merged, and then diagnostic methods as described herein are carried out on the merged droplet set. Microfluidic devices/systems disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In an embodiment, soft lithography in PDMS may be used to prepare the microfluidic devices. For example, a mold may be made using photolithogrophy which defines the location of flow channels, valves, and filters within a substrate. The substrate material is poured into a mold and allowed to set to create a stamp. The stamp is then sealed to a solid support, such as but not limited to glass. Due to the hydrophobic nature of some polymers, such as PDMS, which absorbs some proteins and may inhibit certain biological processes, a passivating agent may be necessary. Suitable passivating agents are known in the art and include, but are not limited to, silanes, parylene, n-Dodecyl-b-D-matoside (DDM), Pluronic, Tween-20, other similar surfactants, polyethylene glycol (PEG), albumin, collagen, and other similar proteins and peptides.

In systems described herein may further be incorporated into wearable medical devices that assess biological samples, such as biological fluids, of a subject outside the clinic setting and report the outcome of the assay remotely to a central server accessible by a medical care professional.

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for introducing and extracting of fluids into and from the device. The devices may be connected to fluid flow actuators that allow directional movement of fluids within the microfluidic device.

As shown herein the elements of the system are stable when freeze dried, therefore embodiments that do not require a supporting device are also contemplated, i.e. the system may be applied to any suitable surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

In certain embodiments, the CRISPR effector protein is bound to each discrete volume in the device. Each discrete volume may comprise a different guide RNA specific for a different target molecule. In certain embodiments, a sample is exposed to a solid substrate comprising more than one discrete volume each comprising a guide RNA specific for a target molecule. Not being bound by a theory, each guide RNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, the CRISPR effector protein is bound by an antibody specific for the effector protein.

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

The present invention may be used with a wireless lab-on-chip (LOC) diagnostic sensor system. In certain embodiments, the present invention is performed in a LOC controlled by a wireless device (e.g., a cell phone, a personal digital assistant (PDA), a tablet) and results are reported to said device. Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type of RFID tag does not contain an internal power source, but is powered by radio frequency signals received from the RFID reader. Alternatively, the active type of RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input, or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In an embodiment, the wireless device also controls the separation and control of the microfluidics channels for more complex LOC analyses. In an embodiment, a LED and other electronic measuring or sensing devices are included in the LOC-RFID chip. Not being bound by a theory, it is believed that this technology is disposable and allows complex tests that require separation and mixing to be performed outside of a laboratory.

In certain embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device.

In certain embodiments, other methods may be used that rely on RFID or Bluetooth as the basic low-cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay.

The present disclosure is illustrated and further described in more detail with reference to the following non-limiting results and examples.

### EXAMPLES

The inventors have developed ultrasensitive and highly specific detection tool based on CRISPR/Cas technology (CRISIPR is an abbreviation of Clustered Regularly Interspaced Short Palindromic Repeats which binds to a protein named Cas13a) for the direct detection of unamplified nucleic acids and proteins in clinical specimens using 3D printed chips and 3D printed 96 microtiter plates. This method can be applied to identify biomarkers of different diseases such as nucleic acids and/or proteins from eukaryotic or non-eukaryotic sources.

In the example shown below, Alzheimer's disease (AD) was used as a model for testing the efficiency and specificity of the developed assay platform, which was used for the direct and specific detection of unamplified circulating miRNAs and proteins overexpressed and increased in AD directly in plasma clinical samples.

### Example 1: Alzheimer's Disease

Alzheimer's disease (AD) is a neurodegenerative disorder which is the most common type of dementia in aging groups. Patients with AD suffer from progressive symptoms of memory loss, and neural dysfunction. The number of AD patients is currently more than 5 million in the US, and 35 million worldwide and is expected to reach more than 100 million by 2050. Unfortunately, no cure has been proven effective and no drug was approved by the food and drug administration (FDA) for Alzheimer to date. However, the early detection of Alzheimer is very critical as it allows better control of the disease on its onset which significantly slows down the progress of AD and improves patient's quality of life. Early detection of AD currently relies on the detection of proteins in cerebrospinal fluids (CSF) which are beta-amyloid peptide (Aβ) and phosphorylated Tau proteins that require an invasive lumbar puncture procedure for sample collection that limits their use and effectiveness. These proteins can also be measured in serum but their levels may not be table influencing their use alone as biomarkers for early AD. Other imaging techniques such as positron emission tomography (PET) and CT scans may be used only to rule out other reasons such as brain cancer which may cause similar symptoms as early AD. Recently, microRNA (miRNA) has received great attention as biomarkers for early disease detection. Measuring panels of miRNA can detect early AD with 95% confidence levels.

MicroRNA (miRNA) as biomarkers for early AD diagnosis: miRNA is a short noncoding RNA sequence which plays a major regulatory role for gene expression by inhibiting the protein synthesis at the level of the post-transcription. Recently blood circulating miRNA have been widely investigated as biomarkers for AD taking into account that about 70% of the currently discovered miRNAs are expressed in the brain and involved in the neurological processes. Therefore, the development of highly sensitive and selective biosensor for detecting miRNA levels in serum and/or plasma is highly needed as non-invasive method for the early detection of Alzheimer disease.

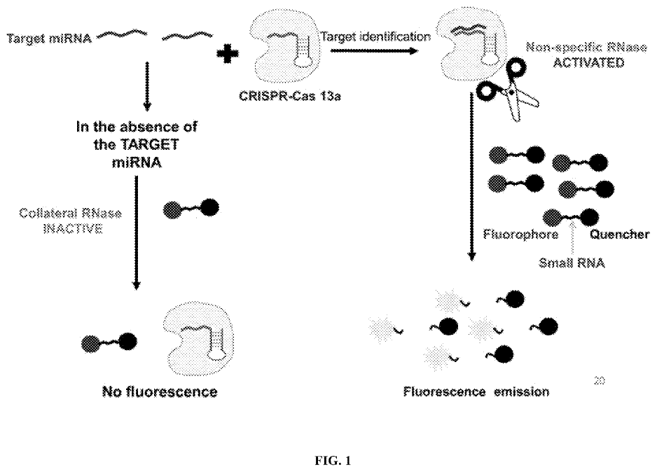

CRISPR as a powerful tool for the detection of miRNA (Fluorescent based signal output): In this sensing platform, CRISPR-cas13a is first programed with the sequence complementary to the target miRNA (named guide RNA or CRISPR RNA, crRNA). This enables the CRISPR-cas13a to identify and bind to only the target miRNA sequence with high specificity. Once the binding is established, the cas13a protein does a conformational changes and act as RNAase protein and cleave the target RNA (cis cleavage). Furthermore, it exhibits non-specific collateral cleavage activity of the surrounding non-target RNAs (FIG. 1).

When the target miRNA sequence is present in the blood sample, the CRISPR-cas13a will identify the sequence, develop the collateral activity (Non-specific RNase activity), and cut a previously added reporter molecule in which a fluorophore and a quencher are linked by small RNA sequence leading to fluorescence emission (FIG. 1). In absence of the target miRNA sequence, the CRISPR-cas13a will not exhibit the collateral effect, and hence the reporter molecule will not be cleaved, and no fluorescence signal is expected. This type of CRISPR-cas13a sensing can be programed to detect any RNA molecule by simply designing the crRNA (guide RNA) with a sequence complementary to the target RNA needed to be tested.

In this developed sensing platform, the detection of 3 miRNA biomarkers were selected for the early detection of Alzheimer's disease. However, this approach can be generalized for the detection of any RNA biomarkers of eukaryotic or prokaryotic source.

### Example 2: Multiplexed Detection of Three miRNAs by Using CRISPR/Cas13a (Fluorescent Assay)

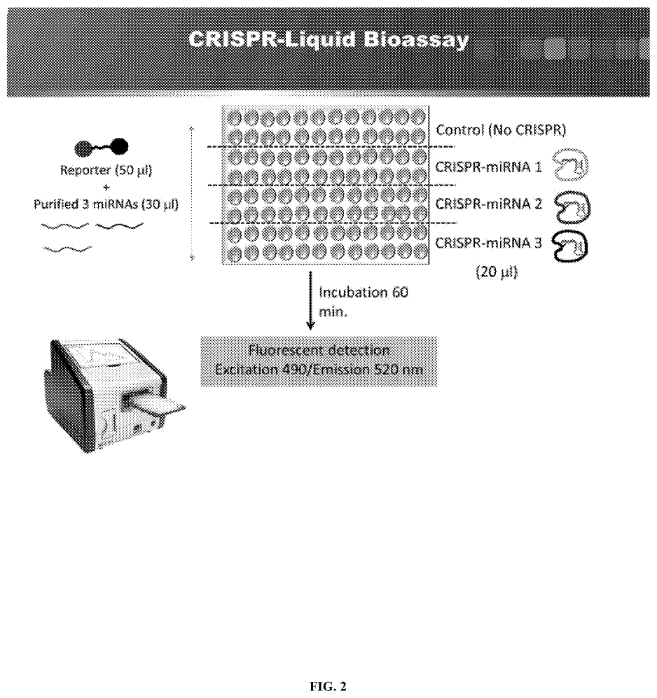

Three blood circulating miRNAs that are correlated to early stage of Alzheimer disease were selected for this multiplexed biosensor development. These miRNA biomarkers are hsa-miR-34c-5p, hsa-miR-30e-5p, and hsa-miR-200c-3p (Table 1). The overexpression and thus, increase in these miRNAs concentration in blood has been linked to the early stage of Alzheimer disease as revealed by studies on human serum. For multiplexing detection of the 3 miRNAs in the conventional assay setup, the pre-designed crRNA for each miRNA is added in the detection chamber along with the cas13a protein and the cleavage buffer. Finally, the reporter molecule will be added to the detection well to develop the florescence signal (FIG. 2).

The invented platform relies firstly on immobilization of the crRNA/cas13a nucleoprotein complex in the microwell of 3D printed 96 microtiter plate for the first time. Then, the reporter mixture (cleavage buffer, RNase inhibitor, RNA reporter molecule) is added along with the analyte sample, and the generated signal is recorded (FIG. 3). For Multiplexing of different analyte (3 miRNAs as a model) in the same run, the cas13 and the crRNA specific for each target are immobilized in the plate. As shown in FIG. 3, the cas13a protein with the corresponding crRNA is immobilized in specific row, where different concentrations of each target will be added in each well to generate a standard curve and three wells for the same sample (triplicate), and two wells for the control (negative control). Thus, the one 3D printed 96 well microtiter plate can be used for analyzing several samples, standard curve, and controls at the same time. Also, the immobilized CRISPR/cas nucleoprotein is highly stable and can be used as a ready-made plate for many samples.

TABLE 1
Selected microRNAs biomarkers

| miRNA name | Sequence |
|---|---|
| hsa-miR-34c-5p | 5′-AGGCAGUGUAGUUAGCUGAUUGC-3′ |
| hsa-miR-30e-5p | 5′-UGUAAACAUCCUUGACUGGAAG-3′ |
| hsa-miR-200c-3p | 5′-UAAUACUGCCGGGUAAUGAUGGA-3′ |

TABLE 2
Summary of the calibration curve for the three
miRNAs targets (Fluorescent Detection)

| miRNA Biomarker | Linear Dynamic Range | LOD (fg/mL) |
|---|---|---|
| 34c-5p | 0.74 fg/mL-740 pg/mL | 0.74 |
| 30e-5p | 0.70 fg/mL-1756 pg/mL | 0.70 |
| 200c-3p | 7.4 fg/mL-74 pg/mL | 7.4 |

The fluorescent based developed platform has been applied to detect the three selected miRNAs in spiked serum and plasma which yield a recovery of 88% to 119%. A broad dynamic linear range has been obtained ranging from 1.8 picogram/ml to 740 attogram/ml. The developed biosensor could sense down to about 0.035 fg per reaction (about 2500 molecules) without pre-amplification step.

### Example 3: Multiplexed Detection of Three miRNAs by Using CRISPR/Cas13a (Electro-Chemiluminescent Based Signal—ECL)

The invented ECL based platform is based on utilizing the redox reaction between Ruthenium bipyridyl poly vinyl pyridine [Ru(bpy)₂ (PVP)₁₀]⁺² and guanine nucleotides. The principle of the reaction pathway is shown in equations 1-4.

Ru^{II}-PVP is oxidized by the electrode (Eq. (1)) at the specified volt and in turn oxidizes guanines nucleotide (Eq. (2)). The guanine radical formed can be further oxidized by Ru^{III}-PVP to give the photoexcited Ru^{II*}-PVP (Eq. (3)) which emits light at 610 nm upon electronic relaxation (Eq. (4)). The developed assay exploiting the collateral cleavage activity of cas13a upon hybridization of the crRNA with the target RNA, by introducing synthesized poly-Guanines (of RNA nature) into the reaction mixture, and in the presence of the target, the non-specific collateral cleavage activity of cas 13a is turned on and cas13a cleaves the poly G generating single guanine nucleotides making them more accessible to the Ru-PVP immobilized on the working electrode and ECL signal is generated due to the reaction pathway shown below (equations 1-4), (FIG. 4).

$${Ru}^{II} - PVP ↔ Ru III - PVP + {e}^{-} ( at electrode ) \qquad ( 1 )$$

$${Ru}^{III} - PVP + G \to Ru II - PVP + {G}^{} + {H}^{+} \qquad ( 2 )$$

$${G}^{} + {Ru}^{III} - PVP \to G 2 ox + {Ru}^{II *} - PVP \qquad ( 3 )$$

$${Ru}^{II *} - PVP \to {Ru}^{II} - PVP + hv ( 610 nm ) \qquad ( 4 )$$

The developed platform (Electro chemiluminescent based signal) is designed by utilizing for the first time the redox reaction between Ruthenium bipyridyl poly vinyl pyridine [Ru(bpy)₂ (PVP)₁₀]⁺² and guanine nucleotides in generating ECL signal by CRISPR/cas technology. This is achieved by firstly construction a layer by layer assembly of poly[diallyl dimethyl ammonium chloride](PDDA), polystyrene sulfonate (PSS) and Ru PVP on pyrolytic graphite sheet as working electrode (FIG. 4B) and then, reaction buffer, RNase inhibitor, cas13a crRNA, crRNA are mixed, incubated at 37° C. for 12 minutes and then, poly r Guanine (having ribonucleic acid backbone, poly r G), and the sample are added and the ECL signal imaged by CCD camera (FIG. 4C). The principle of the detection is that in the presence of the target, Cas protein non-specific collateral property will be activated, which in turn will cleave the poly r G generating single guanine nucleotides which will be oxidized by Ru⁺³ and the photo excited Ru⁺² is generated that emits light at 610 nm upon electronic relaxation.

TABLE 3
Summary of the calibration curve for the
three miRNAs targets (ECL Approach)

ECL Method

| Type of marker | Linear Range | LOD | |
|---|---|---|---|
| miR 34c-5p | 7.4 pg/ml-47 ug/ml | 0.074 | fg/ml |
| miR 30e-5p | 70 pg/ml-7 ug/ml | 42 | pg/ml |
| miR 200c-3p | 7.4 fg/ml-74 ug/ml | 0.15 | fg/ml |

The ECL based developed platform has been applied to detect the three selected miRNAs in spiked serum and plasma which yield a recovery of about 88% to 119%. A broad dynamic linear range has been obtained ranging from 7 microgram/ml to 740 attogram/ml. The developed biosensor could sense down to about 0.035 fg per reaction (about 300 molecules) without pre-amplification step.

### Definitions

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” and “protein” have their common meanings as would be understood by a biochemist of ordinary skill in the art. Terms “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). Standard single letter nucleotides (A, C, G, T, U) and standard single letter amino acids (A, C, D, E, F, G, H, I, K, L, M, N, P, Q, R, S, T, V, W, or Y) are used herein. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholine nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any suitable length, either deoxyribonucleotides or ribonucleotides, or analogs thereof.

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or a RNA chain that has functional role to play in an organism. For the purpose of this disclosure it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

A sequence of bases is a succession of bases signified by a series of a set of five different letters that indicate the order of nucleotides forming alleles within a DNA (using GACT) or RNA (GACU) molecule. By convention, sequences are usually presented from the 5′ end to the 3′ end. For DNA, the sense strand is used. Because nucleic acids are normally linear (unbranched) polymers, specifying the sequence is equivalent to defining the covalent structure of the entire molecule.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “²% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human or a non-human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. Treatment means any way the symptoms of a pathological condition, disorder, or disease are ameliorated or otherwise beneficially altered. Preferably, the subject in need of such treatment is a mammal, preferably a human. Treatment also means providing an active compound to a patient in an amount sufficient to measurably reduce pathological symptoms disclosed herein, slow pathological condition or cause pathological condition regression. These terms also encompass therapy and cure.

As used herein, the term “effective amount” or “therapeutically effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose. As used herein, the term “prophylaxis” refers to preventing or reducing the progression of a disorder, either to a statistically significant degree or to a degree detectable by a person of ordinary skill in the art.

As used herein, the term “dose” or “dosage” denotes any form of an active ingredient formulation or composition, including cells, that contains an amount sufficient to initiate or produce a therapeutic effect with at least one or more administrations. “Formulation” and “composition” are used interchangeably herein. As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments. The terms “subject” and “patient” are used interchangeably herein. The term “subject” also refers to an organism, tissue, cell, or collection of cells from a subject.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A significant change is any detectable change that is statistically significant in a standard parametric test of statistical significance such as Student's T-test, where p<0.05.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims. Any combination of the above-described elements in all possible variations thereof is encompassed by the disclosure unless otherwise indicated herein or otherwise clearly contradicted by context.

For reasons of completeness, various aspects of the disclosure are set out in the following numbered clauses:

Clause 1. A system for detecting one or more biomarkers in a biological or environmental sample, the system comprising: (a) a component configured to contain a plurality of CRISPR-associated (Cas) effector proteins and corresponding guide RNAs, and to receive the sample; and (b) a detector configured to detect a signal generated by activity of the Cas effector proteins in response to a target biomarker in the sample, wherein the signal comprises a fluorescence signal or an electrochemiluminescence (ECL) signal, and wherein detection of the biomarker is performed without nucleic acid amplification.

Clause 2. The system of Clause 1, wherein the biomarkers comprise nucleic acids, proteins, peptides, antibodies, or combinations thereof.

Clause 3. The system of Clause 1, wherein the Cas effector proteins comprise Cas13a, Cas12a, Cas14, or combinations thereof.

Clause 4. The system of Clause 1, wherein the component comprises a microtiter plate having a plurality of discrete wells.

Clause 5. The system of Clause 4, wherein the microtiter plate is formed by additive manufacturing using a 3D printer.

Clause 6. The system of Clause 4, wherein Cas effector proteins and corresponding guide RNAs are immobilized within the wells.

Clause 7. The system of Clause 1, wherein the detector comprises a fluorescence plate reader.

Clause 8. The system of Clause 1, wherein the detector comprises an electrochemiluminescence detector.

Clause 9. The system of Clause 8, wherein the ECL signal is generated using a ruthenium-containing electrochemiluminescent compound.

Clause 10. The system of Clause 1, wherein the component is configured to enable multiplex detection of a plurality of different biomarkers from the same sample.

Clause 11. A diagnostic device for detecting biomarkers, comprising: (a) a 3D-printed substrate defining a plurality of reaction sites; (b) CRISPR-associated effector proteins and corresponding guide RNAs immobilized at the reaction sites; and (c) an optical or electrochemical interface configured to detect fluorescence or electrochemiluminescence (ECL) generated at the reaction sites in response to target biomarkers.

Clause 12. The device of Clause 11, wherein the reaction sites comprise wells of a 96-well microtiter plate.

Clause 13. The device of Clause 11, wherein the CRISPR-associated effector proteins are immobilized via covalent coupling to the substrate.

Clause 14. The device of Clause 11, further comprising an electrochemical electrode assembly associated with each reaction site.

Clause 15. The device of Clause 14, wherein the electrode assembly comprises a pyrolytic graphite electrode coated with a multilayer film including a ruthenium bipyridyl polymer.

Clause 16. The device of Clause 11, wherein the device is configured to generate an ECL signal via collateral cleavage of a poly-guanine RNA substrate by an activated Cas effector protein.

Clause 17. The device of Clause 11, wherein the device is configured for detection of proteins using an RNA-labeled affinity reagent.

Clause 18. The device of Clause 17, wherein the affinity reagent comprises an antibody, nanobody, aptamer, or combination thereof.

Clause 19. The device of Clause 11, wherein the device is integrated into a microfluidic diagnostic cartridge.

Clause 20. The device of Clause 11, wherein the device is configured for point-of-care or portable diagnostic use.

Clause 21. A method for detecting one or more biomarkers, comprising: (a) contacting a sample with a plurality of CRISPR-associated effector proteins and corresponding guide RNAs immobilized on a substrate; (b) allowing a target biomarker to activate at least one of the Cas effector proteins; and (c) detecting a fluorescence signal or an electrochemiluminescence (ECL) signal generated as a result of Cas-mediated cleavage, without nucleic acid amplification.

Clause 22. The method of Clause 21, wherein the biomarker is a nucleic acid biomolecule present in plasma, serum, saliva, urine, or cerebrospinal fluid.

Clause 23. The method of Clause 21, wherein the biomarker is a protein biomarker.

Clause 24. The method of Clause 23, further comprising binding the protein biomarker using a capture reagent and introducing an RNA-labeled secondary binding reagent.

Clause 25. The method of Clause 21, wherein the Cas effector protein exhibits collateral RNase activity.

Clause 26. The method of Clause 21, wherein detecting the signal comprises measuring electrochemiluminescence generated by oxidation of guanine nucleotides.

Clause 27. The method of Clause 26, wherein the guanine nucleotides are generated by Cas-mediated cleavage of a poly-guanine RNA substrate.

Clause 28. The method of Clause 21, wherein the method enables multiplex detection of a panel of disease-associated biomarkers.

Clause 29. The method of Clause 21, wherein the method is performed in a 3D-printed microtiter plate.

Clause 30. The method of Clause 21, wherein the method detects biomarkers associated with neurodegenerative disease, cancer, cardiovascular disease, or infectious disease.

Clause 31. The method of Clause 21, wherein detection occurs within 30 minutes of sample introduction.

Clause 32. The method of Clause 21, wherein the method achieves femtogram-per-milliliter sensitivity.

Clause 33. The method of Clause 21, further comprising generating a standard curve using varying concentrations of a control analyte.

Clause 34. The method of Clause 21, wherein different guide RNAs are spatially separated to detect different biomarkers in parallel.

Clause 35. The method of Clause 21, wherein the method is performed without thermal cycling.

Clause 36. The method of Clause 21, wherein the signal is recorded using a CCD camera or photodetector.

Clause 37. The method of Clause 21, wherein the sample volume is less than 100 microliters.

Clause 38. The method of Clause 21, wherein the method is implemented in a microfluidic device.

Clause 39. The method of Clause 21, wherein the method is compatible with freeze-dried reagents immobilized on the substrate.

Clause 40. The method of Clause 21, wherein the method is used for monitoring disease progression or therapeutic response.

Clause 41. A kit for detecting one or more biomarkers in a sample, the kit comprising: (a) a substrate defining a plurality of assay regions, wherein the assay regions comprise a CRISPR effector protein and corresponding guide nucleic acid or are configured to receive the same; (b) a reporter composition for fluorescence or electrochemiluminescence detection; and (c) instructions for using the kit to detect one or more biomarkers in a sample.

Clause 42. The kit of Clause 41, wherein the substrate is a 3D-printed microtiter plate

Clause 43. The kit of Clause 41, wherein the kit further comprises buffer, nuclease inhibitor, calibration standards, magnetic beads, nanoparticles, capture reagents, and sample-processing reagents.

Clause 44. The kit of Clause 41, wherein the kit is configured for detection of a plurality of biomarkers in parallel.

Clause 45. The kit of Clause 41, wherein the biomarkers comprise disease-associated miRNAs.

Clause 46. The kit of Clause 41, wherein the biomarkers comprise one or more proteins detected through a nucleic-acid trigger strategy.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A system for detection of biomarkers of clinical disorders and infectious diseases, the system comprising:
a component configured to house multiple CRISPR proteins and at least one sample; and
a plate reader configured to detect fluorescence or electrochemiluminescence (ECL) from a reaction between the proteins and the at least one sample, without the need for any amplification, wherein the fluorescence or ECL is an indication of a biomarker of a clinical disorder or an infectious disease.

2. The system of claim 1, wherein the biomarkers comprise at least one of: nucleic acids, proteins, antibodies, or small peptides.

3. The system of claim 1, wherein the CRISPR proteins comprise at least one of Cas13a, Cas12a, or Cas14.

4. The system of claim 1, wherein the component comprises pyrolytic graphite electrodes coated with polyion films containing a ruthenium complex.

5. The system of claim 1, wherein the component comprises a microtiter plate.

6. The system of claim 5, wherein the microtiter plate comprises 96 wells.

7. The system of claim 5, wherein the microtiter plate is a 3D microtiter plate and wherein the microtiter plate comprises the CRISPR proteins immobilized on the 3D printed microtiter plate.

8. The system of claim 7, wherein the microtiter plate is configured to house a CRISPR RNA corresponding to the CRISPR proteins, a buffer, a RNase inhibitor, and a fluorescent reporter.

9. The system of claim 8, wherein the plate reader is configured to detect fluorescence produced by the fluorescent reporter.

10. The system of claim 5, wherein each row of the microtiter plate includes one sample, a concentration of the sample increasing across each row.

11. A method for detection of biomarkers of clinical disorders and infectious diseases, the method comprising:
collecting a sample from a subject;
housing the sample within a component configured to house multiple CRISPR proteins;
performing RNA extraction to get extracted RNA from the sample; and
analyzing the extracted RNA using a plate reader configured to detect fluorescence or electrochemiluminescence (ECL) from a reaction between the proteins and the sample, without the need for any amplification, wherein the fluorescence or ECL is an indication of a biomarker of a clinical disorder or an infectious disease.

12. The method of claim 11, wherein the sample comprises 0.1-10% diluted plasma or serum.

13. The method of claim 11, further comprising capturing and separating a target protein with a magnetic bead or monoclonal antibody.

14. The method of claim 13, further comprising resuspending the target protein with a nanoparticle functionalized with a secondary antibody and a RNA strand.

15. The method of claim 11, wherein the biomarkers comprise at least one of: nucleic acids, proteins, antibodies, or small peptides.

16. The method of claim 11, wherein the CRISPR proteins comprise at least one of Cas13a, Cas12a, or Cas14.

17. The method of claim 11, further comprising producing the component with a 3D printer.

18. The method of claim 17, wherein the component comprises a microtiter plate, the microtiter plate produced with the CRISPR proteins immobilized on the microtiter plate.

19. The method of claim 11, wherein the RNA extraction is performed with a CRISPR RNA corresponding to the CRISPR proteins, a buffer, a RNase inhibitor, and a fluorescent reporter.

20. The method of claim 19, wherein the plate reader is configured to detect fluorescence produced by the fluorescent reporter.
